(19) United States
(12) Patent Application Publication
(10) Pub. No.: US 2011/0118608 A1
(43) Pub. Date: May 19, 2011

(54) SYSTEM AND METHOD FOR DETECTION OF A TEMPERATURE ON A SURFACE OF A BODY

(76) Inventors: Bjoern LINDNER, Aachen (DE); Juergen Philipps, Aachen (DE); René Beaujean, Aachen (DE)

(21) Appl. No.: 12/876,645

(22) Filed: Sep. 7, 2010

(30) Foreign Application Priority Data
Sep. 4, 2009 (DE) 102009040017

(51) Int. Cl. A61B 6/00

(52) U.S. Cl. 600/474

(57) ABSTRACT

A system and method for detection of a temperature on a surface of a body is provided. The system includes a first IR camera system via which the temperature of a certain area of the surface is detectable in the form of a thermal image; a second camera system via which three-dimensional information is detectable regarding a position of a specific area of the body surface related to the first IR camera system; at least a first database in which at least for one body a corresponding model is stored by which at least one contour of the body is defined; and an analyzing unit connectable with the first database receiving the thermal image detected by the first IR camera system and the 3D information detected by the second camera system. Whereby the analyzing unit correlates the 3D information detected by the second camera system with a selectable model stored in the first database thereby generating a data record, which contains exclusively 3D information allocated to the model, the 3D information of the data record subsequently being transformed into a 2D space, and wherein the transformed 2D information of the generated data record is linkable to the thermal image so that exclusively such pixels of the thermal image are selected corresponding to the 2D information of the data record.

This nonprovisional application claims priority under 35 U.S.C. §119(a) to German Patent Application No. DE 102009040017, which was filed in Germany on Sep. 9, 2009, and which is herein incorporated by reference.

### BACKGROUND OF THE INVENTION

1. Field of the Invention

The present invention relates to a system and method for detection of a temperature on a surface of a body of any shape whatsoever, in particular for recognition of decubitus wounds on the human body or for recognition of temperature increases within the scope of an infectious and/or virus disease, for example in the event of a H1N1 or H5N1 infection.

2. Description of the Background Art

In many fields it is important to detect the temperature on the surface of technical devices, machines, industrial plants or buildings. The same applies to the examination of human or animal bodies where areas of different temperature may occur on the skin, in particular if sore locations have formed.

On the human body, decubitus wounds occur on the skin and the subjacent connective tissue due to long-lasting mechanical stress and reduced perfusion. Often bedridden patients are affected or individuals sitting in a wheelchair for a long time. By trend, the number of patients developing such decubitus wounds increases with the number of bedridden patients. In aging societies as they exist today in many industrialized countries the number of aged individuals in hospitals or residential care homes increases. During these stages in particular longer times in bed are often indicated. The obvious means for avoiding decubitus wounds is frequent relocation of patients by the nursing staff. Due to the tendency for cost reduction in the nursing staff field, the management of a care facility has a great organizational responsibility. If a decubitus wound occurs on a patient, the individuals concerned or third parties may possibly deduce failure by the nursing staff from it. Moreover, care for healing of these wounds is associated with considerable cost for the care unit. If a patient is relocated from one establishment into another, the nursing management of the releasing establishment for the above reasons is interested in proving that the patient has left without decubitus in order not to be held responsible later. Likewise, the management of the accepting establishment is interested in checking whether the patient already has an existing or arising decubitus so that it can be proven that it is not their responsibility. In any case it is important for every care facility to recognize and classify a decubitus on their patients already at a very early stage of formation so that treatment can start early. Successful treatment must also be checked in regular intervals in order to be able to adjust the method of treatment, if required.

From literature it is known that decubitus wounds due to metabolic processes differ from the surrounding healthy skin by a characteristic temperature pattern. In the study Development of a Thermal and Hyperspectral Imaging System for Wound Characterization and Metabolic Correlation” , M.H. Chen at al., JOHNS HOPKINS APL TECHNICAL DIGEST, VOLUME 26, NUMBER 1/2005, it has been proven that by means of a thermal image camera, digital recordings of the skin can be made permitting identification of decubitus wounds by means of subsequent manual estimation by an experienced medial expert. For the purpose of improved representation, in the same reference also use of stereo cameras for generation of a 3D image of the wound area is described. The stereo images produced in this context are not evaluated algorithmically but rather submitted by means of an appropriate display unit (e.g. a 3D monitor or by means of 3D glasses) to a human expert for spatial examination.

A related subject is the detection of skin lesions caused by skin cancer. From U.S. 2008/0226151 a portable device for examination of skin lesions is known. In the case of this device the cameras used can be realized not only as thermal image cameras but also as normal cameras in visible light. Alternatively, this device may also have a stereo image camera operated in 3D mode. Irrespective of the different camera types, the object of this device is only three- dimensional reconstruction and representation of a wound.

Although with the device according to U.S. 2008/0226151, automatic recognition and evaluation of a wound on the skin is supposed to be possible, from the design of the device and the operating instructions it can be taken by the average expert, however, that the medical practitioner as soon as he or she has recognized a wound must bring the device close to the supposed wound in order to make a recording of the wound. This means that primary recognition of the wound must be made manually by the medical practitioner itself.

According to conventional automatic detection of wounds is possible—if at all—only in such a way that a thermal image sensor is brought very close to the body surface. In this connection it has to be taken care that interfering objects in the thermal image, which might exist in the background of the body to be examined, should not be included in the recording since otherwise errors of measurement and/or pixels in the thermal image, which are not allocable, may occur. Without knowledge as to where in the thermal image the actual body surface to be examined is located, and where in the thermal image interfering objects exist, many pseudo-detections of wounds occur in an automatic image representation and/or evaluation. In that case a human operator (medical practitioner) must subsequently decide manually and on an individual case basis whether an actual wound exists or whether an interfering object in the image background of the thermal image camera has caused a pseudo-detection instead. This manual decision results in the disadvantages that preparation of the thermal image always required very skilled operating personnel and also requires a long scan time.

### SUMMARY OF THE INVENTION

It is therefore an object of the present invention to reliably detect the temperature on a surface of a body without external interfering objects in the background of the body falsifying the measuring result.

An inventive system serves for detection of a temperature on a surface of a body and comprises a first IR camera system by means of which the temperature of a certain area of the surface is detectable in the form of a thermal image, a second camera system by means of which three-dimensional (3D) information is detectable regarding the position of the specific area of the body surface related to the first camera system, at least a first database in which at least for one body a corresponding model is stored by which at least one contour of the body is defined, and an analyzing unit connected with the first database receiving the thermal image detected by the first IR camera system and the 3D information detected by the second camera system. The analyzing unit correlates the 3D information detected by the second camera system with a selectable model stored in the first database, thus generating a data record, which contains exclusively 3D information unequivocally allocable to the model, with the 3D information of the data record subsequently being appropriately transformed into a 2D space. The transformed 2D information of the generated data record are linked to the thermal image so that exclusively such pixels of the thermal image are selected corresponding to the 2D information of the data record. Such linking may either be carried out by the analyzing unit or by another appropriate data processing device.

By means of the inventive system it is possible for an operator to prepare a thermal image of the body surface in its natural environment with it being possible that different other objects are located in proximity or in the background without measuring errors or similar occurring. If the body is a human patient, a thermal image of the patient can be taken at the bedside or in the sickroom. Whereas by means of the first IR camera system a thermal image of a specific area of the body surface is taken, at the same time by means of the second camera system three-dimensional (3D) information concerning the position of this specific area of the body surface relative to the first IR camera system is detected. The analyzing unit subsequently correlates the 3D information detected by the second camera system with a selectable model stored in the first database by which at least a contour of the body and/or of a specific body area is defined. The data record generated by such correlation contains exclusively 3D information which can unequivocally allocated to the model. This means that all external objects, which might exist in the image background, are filtered out so that these “external” 3D information is not part of the data record. Subsequently the 3D information of the data record is appropriately transformed into a 2D space. An important characteristic of the invention is the fact that the transformed 2D information of the generated data record is linked to the thermal image so that exclusively such pixels of the thermal image are selected which correspond to the 2D information of the data record. This linking results in the fact that for analysis of the thermal image only such pixels are selected, which are unequivocally allocated to the contour of the body to be examined and/or to the area defined by such contour. Accordingly, for analysis of the thermal image all other pixels not belonging to the body to be examined but are traced back to objects in the background instead are not considered thus excluding so-called pseudo-detections of wounds or similar.

As has been explained above, the model stored in the first database defines at least a contour of the body and/or a specific area of the body. This means that the model is described by two-dimensional data or three-dimensional data. Due to the correlation mentioned of the three-dimensional information, which is detected by the second camera system, by means of the analyzing unit it can be determined within a short period of time with the data of the model which type of measuring object the body to be examined is, and what objects in its environment and/or background are not supposed to be considered for this measurement.

Within the meaning of the present invention the database can be understood as any memory suitable for storing data. These data can be read-out from the database in a known manner with it being possible that modified data are stored again in the database. The database may be a memory chip or a local or network-based database with data being read-out and/or written into the database via a data network.

In an embodiment of the invention, the system may comprise a second database in which defined temperature data of the specific area of the body surface are stored. Moreover, this embodiment comprises a recognition module associated with the second database with the temperature data of the selected pixels of the thermal image being comparable to the defined temperature data stored in the second database so that deviations or correlations between the respective temperature data can be determined. The defined temperature data stored in the second database may be, for example, relative temperature data, i.e. temperature deviations typically exhibited by specific zones, e.g. normal and abnormal areas, on the body surface relative to each other. In addition or alternatively, the defined temperature data may also be absolute temperature data, i.e. specific temperatures typically exhibited by a body surface or specific areas of a body surface. Thus, by means of the recognition module e.g. specific temperature patterns can be recognized on the surface of a body, which are characteristic for decubitus wounds.

In an embodiment of the invention, the system may comprise a third VIS camera system by means of which a VIS image of the specific area of the body surface is taken. In the same way as with reference to the thermal image the transformed 2D information of the generated data record can be linked to the VIS image so that exclusively such pixels of the VIS image are selected which correspond to the 2D information of the data record after transformation from the 3D space.

The VIS image is taken by the same specific area of the body surface in respect of which also the thermal image by means of the first IR camera system is taken. The VIS image facilitates for an operator to understand the thermal image generated since the VIS image is known to be a grayscale picture and/or colored picture. By linking the transformed 2D information of the generated data record to the VIS image, also in the latter all external and thus interfering objects in the background of the image are filtered out so that finally exclusively an image of the body to be examined and/or the specific area of its body surface is taken into account for further data evaluation.

In an embodiment of the invention, the third VIS camera system can be integrated into the second camera system so that by means of a camera unit of the second camera system a VIS image of the specific area of the body surface can be taken. In other words, a camera unit of the second camera system may at the same time have the quality to generate a grayscale or colored picture representing a copy of the visible area of the body surface.

In an embodiment of the invention, the system can comprise another (third) database in which the selected pixels of the thermal image and/or the selected pixels of the VIS image and/or the identified and classified zones with abnormal areas can be stored. The same applies with respect to the deviations and/or correlations between the respective temperature data of the selected pixels of the thermal image and the defined temperature data stored in the second database. By storing the above mentioned information in the third database, a plurality of information can be collected, which at least with respect to a specific patient can serve as reference data for corresponding comparing inquiries or similar. In other words, the information stored in the third database can serve for providing comparative recordings and/or values in order to inform for example on the history of a patient's illness.

In an embodiment of the invention, the first IR camera system and the second camera system can be arranged within a common housing which can be carried by an operator. Advantageously the respective cameras of the first and/or second camera system are arranged here collinearly to each other, so that their lenses and/or objectives are mainly in a common plane. The housing is designed such that it can be taken by an operator by one hand in order to make a recording (for example of a patient).

In an embodiment of the invention, the system comprises a distance measuring device by means of which a distance between the specific area of the body surface and the respective camera system(s) can be determined. In other words, by means of this distance measuring device the distance between the body surface and the first and/or second camera system is determined. The distance measuring device may for example be configured as a point-shaped triangulation sensor which is directed towards the specific area of the surface of the body to be analyzed. By an acoustic and/or optical signal it can be displayed whether the second camera system and/or the housing has a correct and/or predetermined distance to the specific area of the body surface. The system is designed such that only after the housing and/or the first IR camera system and the second camera system are positioned in a predetermined distance with respect to the specific area of the body surface, the thermal image suitable for temperature measurement is taken and the 3D information concerning the position of the specific area of the body surface relative to the first IR camera system is detected. Thus, unusable recordings are advantageously avoided and time and effort for the patient's examination is reduced accordingly. A cost-saving manufacture of the system is possible insofar as the distance measuring device is integrated into the second camera system.

In another embodiment, not only the first IR camera system but also the second camera system is in a continuous recording mode. The two camera systems are synchronized insofar as for each 3D information determination of the second camera system a thermal image of the IR camera system can be allocated to time. All thermal images, which due to the three-dimensional position of the housing relative to the body surface -determined by the distance sensor and/or the second camera system- are within suitable distance and position of the device relative to the body surface are registered internally as being usable. Only when the user activates a release button, newly registered thermal images are supplied to subsequent analysis.

In an embodiment of the invention, the first IR camera system may have at least one thermal image camera or a plurality of thermal image cameras. By several thermal image cameras the image range on the body surface can be advantageously enlarged with the measuring data of the individual thermal image cameras being consolidated in an appropriate way in order to finally generate one single thermal image of the body surface. Likewise an embodiment is possible where several body surfaces of different bodies are covered and examined at the same time. This is advantageous, for example, if at an airport gate a plurality of individuals passing a measuring system are measured simultaneously with respect to their temperature (e.g. skin, eyes).

In an embodiment of the invention, the second camera system can at least comprise a Time-of-Flight (TOF) camera and/or at least a stereo vision camera. When a TOF camera is used, the system comprises in addition a device for illuminating the specific area of the body surface with a modulated light the wavelength of which is adjusted to the camera of the second camera system. In this connection the TOF camera determines the runtime of the light for determination of the three-dimensional depth information of each picture element on the body surface. Alternatively the second camera system may comprise one or several so-called stereo vision camera(s) which by simultaneous recording of the body surface from two angles of view and determination of disparities of the individual image contents of the scene automatically determine in the images taken the distance information of the specific area of the body surface relative to the first IR camera system. The stereo camera technique compared with the TOF technique has the advantage that high-resolution color or grayscale cameras can be used which each for itself is a conventional and thus cost-saving standard component as is presently being used in a plurality of consumer equipment. Accordingly, the components for this stereo camera technique can be obtained on a budget-priced level. An improvement can be achieved by illuminating the body surface with a light in a wavelength range to which the stereo vision camera(s) is (are) sensitive.

In an embodiment of the system, a device can be provided to illumnate the specific area of the body surface with colored or white light which can be detected by the third VIS camera system. This improves on the one hand the quality of the VIS image taken and facilitates on the other hand recognition of the recorded specific area of the body surface on an indicating unit (a monitor or similar, for example).

In an embodiment of the invention, the system may provide a battery unit guaranteeing power supply of the individual system components including the various camera equipment. Thus it is ensured that during operation of the system the individual camera equipment is always turned on and has a constant operating temperature. A separate heating up time for the camera equipment is therefore unnecessary.

In an embodiment of the invention, the system can comprise an indicating unit which can show the selected pixels of the thermal image and/or the selected pixels of the VIS image. This has the advantage that the operator immediately after having taken the image of the specific area of the body surface can inspect the images taken by means of the indicating unit. Subsequently the operator can directly decide as to whether more images have to be taken from the specific area of the body surface.

In an embodiment of the invention, by the recognition module an image processing algorithm can be provided by means of which a specific temperature pattern in the form of an “Object-of-Interest” (OOI) can be identified. For example, an OOI may be a decubitus wound. By the image processing algorithm the distance of the OOI to the border of the specific area of the body surface can be determined. In order to guarantee a complete temperature signature and to avoid errors in measurement it is important that in the event of a decubitus wound sufficient skin around the OOI is recorded on the image. Thus, if the distance of the OOI to the border of the contour of the specific area of the body surface is insufficient, an alarm signal is generated by means of an output unit. Advantageously it can also be determined automatically by means of the image processing algorithm in which direction the recording device has to be shifted so that the identified OOI exceeds a requested distance to a border area of the contour of the specific area of the body surface (and/or the transformed 2D data of the data record). In the new position of the recording device a new recording sequence can then be released permitting an improved analysis of the OOI.

A compact design of the system can be easily achieved by integrating the output unit into the indicating unit.

The present invention further provides a method for the detection of temperature on a surface of a body comprising detecting the temperature of a specific area of the body surface in the form of a thermal image by means of a first IR camera system, detecting three-dimensional (3D) information by means of a second camera system regarding the position of the specific area of the surface relative to the first IR camera system, selecting a predetermined model, which defines at least the one contour of the body and is stored in a first database, and subsequent correlating such model by means of an analyzing unit with the 3D information of the specific area of the surface detected by the second camera system with a data record being generated in which exclusively 3D information is contained which is unequivocally allocable to the model, transforming the 3D information of the data record into a 2D space and linking the transformed 2D information of the data record to the thermal image so that only such pixels of the thermal image are selected which correspond to the 2D information of the data record.

This method like the inventive system explained above allows for a filtering out of image information which can exclusively be allocated to the body to be examined. This occurs by correlating the 3D information with the predetermined model of the body to be examined and generation of the data record mentioned which contains solely the 3D information belonging to the body. In other words, all external image information based upon objects in the image background, for example, are omitted and not considered for further data evaluation

After the 3D information has then been suitably transformed into a two-dimensional (2D) space, this transformed 2D information is linked to the thermal image. In the end solely such pixels of the thermal image are then selected which correspond to the 2D information of the data record. Based on a preparation of the 3D information a characteristic of the invention is the fact that only such pixels of the thermal image are considered which can be allocated to the specific area of the body surface.

In an embodiment of the invention, defined temperature data can be provided in the method which are stored in a second database. These temperature data may either be absolute temperature values (for example of specific skin areas) or relative temperature values representing, for example, a specific temperature pattern of adjacent zones of the body surface. In a next step the temperature data of the selected pixels of the thermal image are compared with the defined temperature data stored in the second database so that deviations or correlations between the respective temperature data are determined. In the end it is thus possible to determine increased temperature values in the form of a deviation or specific temperature patterns in the form of a correlation, for example, on a patient's skin.

In an embodiment of the invention, a VIS image can be created in the method of the specific area of the body surface with subsequently the transformed 2D information of the data record generated by the analyzing unit being linked to the VIS image so that exclusively such pixels of the VIS image are selected which correspond to the 3D information of the data record. Generation of a VIS image, which can represent a usual grayscale or colored picture, facilitates for an operator (for example a medical practitioner) perception of the recording of the specific area of the body surface.

In an embodiment of the invention, in the method by means of an image processing algorithm in the specific area of the body surface a specific temperature pattern in the form of an Object-of-Interest (OOI) is identified. An 001 may be a decubitus wound with its characteristic temperature zones. The image processing algorithm determines in addition a distance of the OOI to a border area of the contour of the transformed 2D data of the data record and compares such distance with a predetermined value. If the distance of the OOI to the border area falls below the predetermined value, an alarm signal is generated. This has the advantage that by the generated alarm signal it is indicated to the operator, if the OOI of the thermal image is too close to a border area of the contour which might result in errors or inaccuracies. Advantageously, by the alarm signal a recommended shifting direction for the first IR camera system and/or the second camera system in view of a new recording of the specific area of the surface is shown so that the distance of the identified OOI to the border area of the contour of the transformed 2D data of the data record then exceeds a predetermined distance.

The system and method according to the invention are suitable for creating temperature takings on human or animal bodies for medicinal purposes. This is associated with the following characteristics and/or advantages. In the case of the camera systems used the wavelength of radiation is adjusted in such a way that for example the eyes of a human being are by no means dazzled and/or damaged. This is in particular guaranteed, if the second camera system comprises a TOF camera. The time interval between the takings of the camera systems involved is very short, e.g. 1/10 second. By this it is guaranteed that also in the case of a movement of the body to be measured (e.g. a patient), the takings, which are made by the respective camera systems, can be allocated to each other, because they are mainly based on the identical position of the body and/or the body surface examined. A body to be measured in the form of a human being normally has always two arms and/or two legs formed in a mirror-inverted way to each other. But for an examination only one of these extremities, e.g. the left or right leg and/or the left or right arm is important with the other extremity not being important in each case. In the same way, for example, a body part of a nurse assisting a patient during the taking of an image, is not important for such taking. By the filtering out of external image information mentioned above it is thus possible to reliably suppress interfering extremities of the patient itself or body parts of a nurse from the images of the camera systems so that these are not considered in further image analysis.

Moreover, the invention can be used for measurement of skin or tissue reactions during or after the impact of compressive stress. In addition the system can be used for the detection of bodily infections of a human being or an animal by local temperature measurement on predetermined body locations (for example the inner eye corners in the case of suspected influenza).

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a perspective view of a housing in which substantial components of the inventive system are located;

FIG. 2 is a lateral sectional view of the housing of FIG. 1;

FIG. 3 is a longitudinal sectional view of the housing of FIG. 1;

FIG. 4 is a flowchart for use of the inventive system and/or performance of the inventive method according to a first embodiment;

FIG. 5 is a flowchart for use of the inventive system and/or performance of the inventive method according to a second embodiment; and

FIGS. 6-8 are another embodiment of the inventive system with its housing being shown in a front view, in a lateral sectional view and/or a longitudinal sectional view.

### DETAILED DESCRIPTION

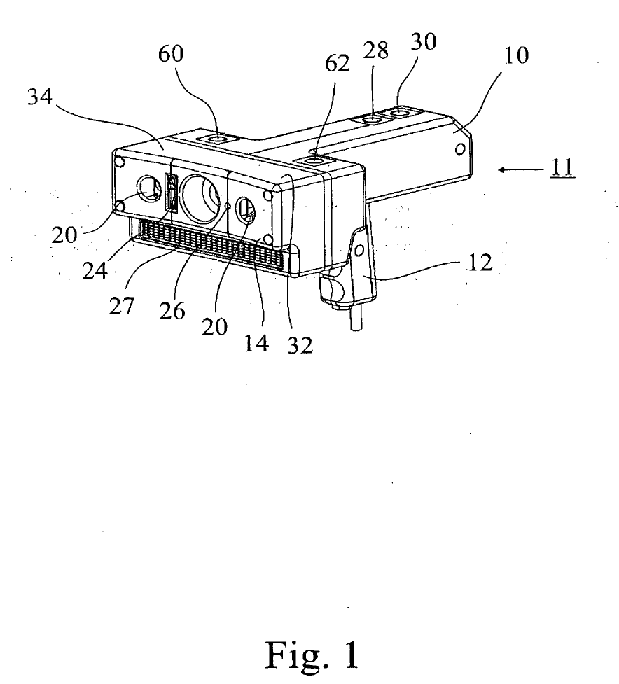

FIG. 1 is a perspective view of a housing 10 accommodating some important components of an inventive system. The housing 10 is of compact design and can be easily handled by hand by an operator. For this, a grip area 12 is formed at the bottom of the housing 10.

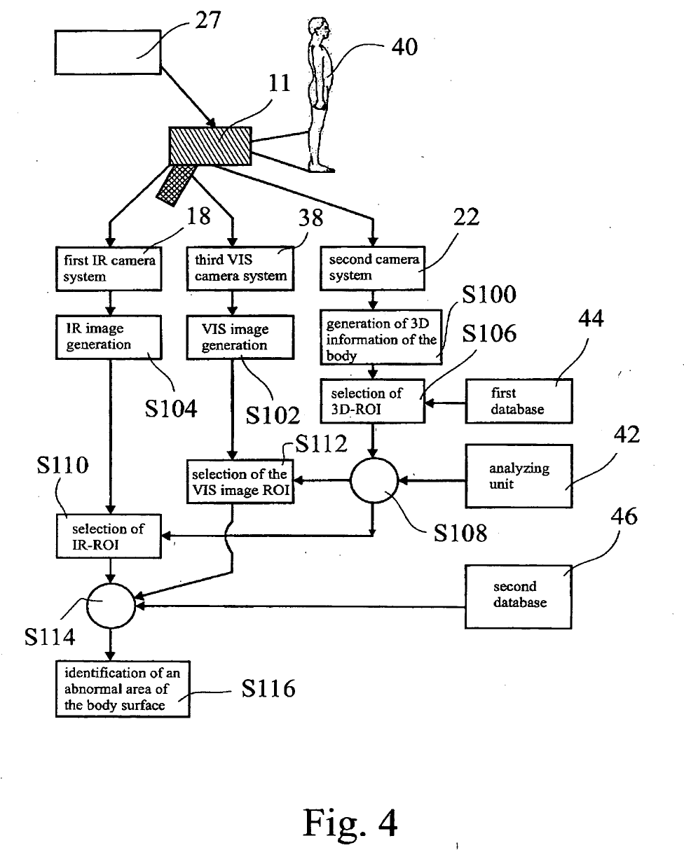

The housing 10 comprises a front face 14 which during an examination is oriented towards a surface of a body to be examined. On the front face 14 a thermal image camera 16 in the form of an IR camera is located in a central area. This thermal image camera 16 is to be understood as a first IR camera system 18. Contrary to the embodiment shown in FIG. 1, the first IR camera system 18 may also comprise a plurality of thermal image cameras 16. Moreover, one stereo camera 20 each is located on the border areas of the front face 14 of the housing 10. The two stereo cameras 20 are part of a second camera system 22. Further, an optical distance sensor 24 and a light spot projector 26 are provided on the front face 14. The optical distance sensor 24 determines a distance between a surface of a body 40 to be analyzed (FIG. 4) and the front face 14 and/or the camera systems 18, 22 mentioned which are located in the same. The light spot projector 26, for example, generates a single point laser beam mainly in line with the beam path of the first and second camera system 18, 22. By means of the light spot projector 26 an operator can visualize to which point the beam paths of the first and/or second camera system 18, 22 are directed so that a “targeting” is facilitated and/or more precise when an image is taken.

The front face 14 is provided in addition with an illumination unit 27 comprised of switchable red and white LEDs. The beam path of these LEDs is mainly in line with the beam paths of the first and second camera system 18, 22 so that a surface of the body 40 to be analyzed is lighted and/or illuminated by the illumination unit 27.

On an upper surface of the housing 10 a front LED 28 and a rear LED 30 are located. Moreover, on the upper surface of the housing 10 a left LED 32 and a right LED 34 is located. Moreover, a right push-button switch 60 and a left push-button-switch 62 are located on an upper surface of the housing. The operating mode of these LEDs 32, 34 and the push-button switches 60, 62 is explained in detail below.

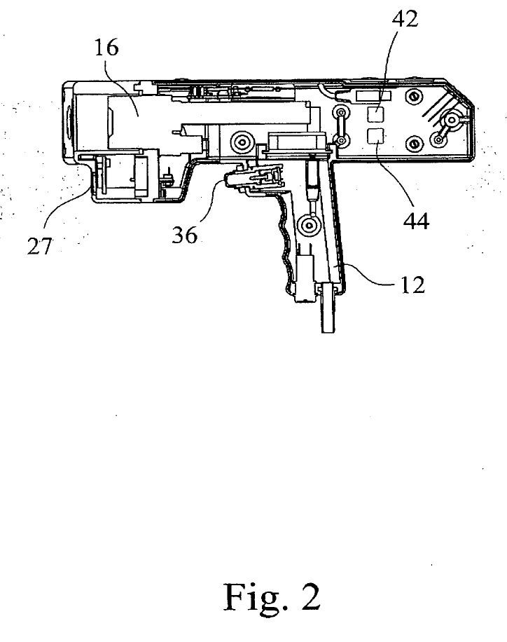

FIG. 2 is a lateral sectional view of housing 10. In an upper portion of the grip area 12 a release button in the form of a key element 36 is provided. By actuating the key element 36, the components of the system located on the front face 14 are activated which will be explained below in detail. In the cross-sectional view of FIG. 2 moreover the thermal image camera 16 and the subjacent illumination unit 27 can be seen.

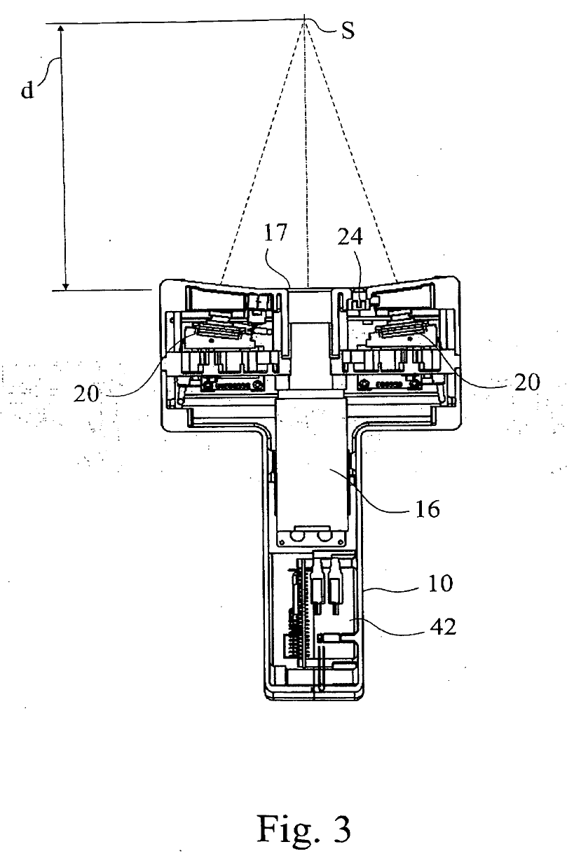

FIG. 3 is a longitudinal sectional view of the housing 10. The thermal image camera 16 is located in a central portion of the housing so that its lens 17 is located accordingly in a central area of the front face 14. To the left and right of the lens 17 the two stereo cameras 20 are located. The cameras 20 are mounted on the housing 10 in an angle such that their beam paths intersect in a common point S in the beam path of the thermal image camera 16. This guarantees that a specific area of the body surface 40 is covered and/or recorded by the thermal image camera 16 and the two stereo cameras 20 at the same time. This intersection S is spaced from the front face 14 at a distance d.

Contrary to the presentation in FIG. 3 it is also possible to mount the two cameras 20 on the housing in such a way that their beam paths relative to each other have another angle than that shown in FIG. 3 or extend mainly parallel to each other.

The two cameras 20 are high-resolution color cameras which are combined into the second camera system 22 as has been explained above. By means of this second camera system 22 according to the known principle of stereo cameras on disparity of picture elements spatial distance of picture elements on a surface of the body 40 to be analyzed relative to the front face 14 and/or the first IR camera system 18 is determined in the two adjacent cameras. In this way, by means of the two stereo cameras 20 three-dimensional (3D) information regarding the position of the specific area of the body surface relative to the first IR camera system can be detected.

The above mentioned stereo cameras 20 moreover serve for generating a VIS image of the specific area of the body surface 40. Such a visual (VIS) image is a usual grayscale or colored picture displaying the surface of the body. In connection with preparation of a VIS image of the specific area of the body surface the stereo cameras 20 are to be understood as part of a third VIS camera system 38. In the embodiment shown in FIGS. 1-3 according to the inventive system the third VIS camera system 38 is integrated into the second camera system.

In the following the housing 10 with its integrated components as shown in FIG. 1 is designated as scanner 1.

In the scanner 11 moreover an analyzing unit 42 and an associated memory chip 44 are disposed shown by simplified symbols in FIG. 2. The memory chip 44 provides a first database in which for at least one body a corresponding model is stored by which at least a two-dimensional contour of the body is defined. In addition, such a model can also define three-dimensional (3D) data of the body. The analyzing unit 42 is logically connected to the first IR camera system 18 and the second camera system 22 so that the generated data (concerning the temperature of a specific area of the body surface with this camera system and the three-dimensional information concerning the position of the specific area of the body surface relative to the first IR camera system 18) are received by the analyzing unit 42. Contrary to a physical arrangement of analyzing unit 42 and memory chip 44 in the scanner 11 it is also possible that their functions are executed by a computer unit to which the scanner 11 is connected.

In FIG. 4 some of the above mentioned components of the inventive system are shown as simplified symbols. In addition, FIG. 4 represents a flowchart for the performance of certain steps on the basis of which the system and/or method according to the invention are realized and/or carried out. Below, use of the system and/or performance of the method are explained in detail with reference to FIG. 4.

The following example describes the use of the invention for recognition of decubitus wounds on a bedridden human patient in a hospital. The patient is understood here as the above mentioned body to be examined and is designated with reference number 40 according to FIG. 4.

At the beginning of a recording, the scanner 11 is brought close to the body part to be examined - in the present example a lower leg of the patient—by an operator (for example, a nurse, a medical practitioner). At first, the operator presses the right push-button switch 60 or the left push-button switch 62, thus generating an information on spatial orientation of the body to be examined. For a partial section of a patient's arm or leg thus by pressing the right or left push-button switch 60, 62 an information on spatial orientation of the body surface of these body parts and their allocation can be generated. Accordingly, a preselection of the model, which is stored in the first database and defines at least a contour of the body and/or a part of it (e.g. lower leg), is possible so that correlation of the 3D information detected by the second camera system with the model and the generation of the mentioned data record based upon it is quicker and more reliable.

While the scanner 11 with its front face 14 is held towards the lower leg, the operator now depresses the key element 36 halfway. By this a point-shaped light beam is generated by means of the light spot projector 26 and projected onto a surface of the lower leg. This indicates to the operator to which location on the lower leg the scanner with its front face 14 and the camera systems provided on it is currently oriented. At the same time, the optical distance sensor 24 measures the distance between the front face 14 (and/or the camera systems provided on it) and the lower leg surface. For preparing a proper recording it is important that this distance is equal to distance d shown in FIG. 3, by taking into account a minor tolerance range, if required. If the actually existing distance between the front face 14 and the surface of the lower leg is situated within the requested range, an acoustic permanent sound is heard. If no permanent sound is heard, this indicates that the distance is too great or too small. Consequently it can be displayed via the front and/or rear LED 28, 30 whether the scanner 11 must be approached closer to the lower leg and/or the distance must be increased. In the same way it can be displayed on the left LED 32 and/or on the right LED 34 whether the scanner 11 has to be located more to the left or more to the right with respect to the lower leg.

After the operator has been informed by the above mentioned acoustic and/or optical signals that distance, position and orientation of the scanner 11 are correct, by a complete pressing of key element 36 the actual recordings by means of the different camera systems 18, 22, 38 are generated and thus the scan mode is started.

At the beginning of the scan mode the point-shaped light beam of the light spot projector 26 is switched off. By means of the stereo camera 20 of the second camera system 22 now at first in step S100 three-dimensional (3D) information concerning the position of the specific area of the lower leg to be examined relative to the front face 14 of the scanner 11 and thus relative to the first IR camera system 18 is detected. In this connection, the red LEDs of the illumination unit 27 are switched on at the same time, thus illuminating the lower leg in red. It has turned out that by this, contrast formation on the surface of the body to be examined (the lower leg in the present case) and thus also the requested three-dimensional information are improved depending on the stereo camera algorithm used.

After having taken a recording for detection of the 3D information, by means of the two stereo cameras 20 in step S102 a VIS image of the lower leg is taken with the red LEDs of the illumination unit 27 being automatically switched off in this process and the white LEDs being activated and/or switched on instead. This means that the lower leg is irradiated with white light while a VIS image or several VIS images are made from the lower leg. In addition by means of the first IR camera system 18 and/or with its thermal image camera 16 in step S104 a thermal image is taken from the lower leg and the temperature on its surface detected during this process.

All of the above mentioned images taken by the camera systems (S100-S104) are suitably stored in the analyzing unit 42 while these images should be taken within a very short time after 3D detection or else almost at the same time. Ideally only a maximum time interval of 1/10 second should be between detection of the 3D information concerning position of the lower leg relative to the first IR camera system 18 and completion of the other recordings. The reason is that otherwise in the event of an involuntary movement by the user or the patient the detected 3D position data of the lower leg would no longer be valid for all recordings.

An important aspect of the invention is that in the image of the lower leg all externally surrounding image areas, which for example originate from external objects in the image background, are suppressed so that these external picture elements have no more influence on the actual image evaluation and the corresponding wound identification and classification. For this purpose, in step S106 a so-called “Region of Interest” (ROI) of the lower leg is selected by correlating by means of the analyzing unit 42 the 3D information detected by the second camera system 22 with a selectable model of the lower leg stored in the first database 44. During this process a data record is generated which contains exclusively 3D information unequivocally allocable to the model of the lower leg. All external picture elements and/or 3D information not belonging to the lower leg are thus filtered out and not taken into account for further image evaluation.

Subsequently in step S108 the 3D information of the data record is suitably transformed into a 2D space by the analyzing unit 42. For this purpose the analyzing unit 42 makes use of a previously produced calibration database containing -and/or computing from predetermined transformation equations- for each point within the three-dimensional space coordinate system of the second camera system 22 the imaging vector in a two-dimensional image space. If the quantity of the identified picture elements contains areas with unidentified picture elements still enclosed in the image space, these holes can be closed by an appropriate interpolation algorithm.

The advantage of the steps S106 and S108 is that at first by the correlation with the model of the body to be examined stored in the first database it can be recognized within a very short period of time of which type the body to be examined is and what picture elements of the recording are to be allocated to the body. In other words, the scanner 11 “recognizes” automatically the type of the body to be examined so that as has been explained above all external picture elements can be filtered out and omitted.

Subsequently to step S108 now in step S110 the transformed 2D information of the data record are linked to the thermal image so that exclusively such pixels of the thermal image are selected, which correspond to the 2D information of the data record, and thus form an Infrared ROI. Hence it is guaranteed that exclusively only such temperature information is taken into account for further evaluation, which belongs to the ROI of the recording of the lower leg, and conversely all other temperature information belonging to external objects are omitted. In the same way the transformed 2D information of the data record in step S112 is linked to the VIS image so that here in the same way as for the IR thermal image a ROI of the VIS image is selected.

In the next step S114 the ROI of the IR image is compared by means of a recognition module with defined temperature data stored in a second database 46. With respect to a decubitus wound such defined temperature data are relative temperature data defining the characteristic temperature levels of adjacent zones of such a decubitus wound. In connection with this comparison the recognition module uses an appropriate image processing algorithm by means of which a characteristic temperature pattern -also called “Object-of-Interest” (OOI)- can be identified in the specific area of the body surface. It shall be understood that an OOI represents an abnormal area and is formed, for example, by a decubitus wound with its characteristic temperature zones. In step S116 it is finally determined whether a correlation of the OOI with a predetermined temperature pattern stored in the second database exists: in the affirmative this means that in the ROI of the IR image an abnormal area of the lower leg is identified and accordingly an OOI in the form of a decubitus wound exists.

The position of an OOI relative to the scanner 11 can be computed by a reverse application of the appertaining transformation vectors. Moreover, the position of an OOI relative to a border area of the appertaining surrounding ROI can be determined. If by the scanner 11 by application of an appropriate algorithm it is recognized that an OOI is too close to the border of an ROI, there is a risk that insufficient skin has been recorded around the OOI in order to cover a complete temperature signature of this potential wound. By means of suitable optical signals the operator can receive corresponding feedback information into which direction he or she has to modify the position and orientation of the scanner 11 in order that the OOI is less close to the border of the ROI. In this new position the operator can then activate a new recording sequence and thus obtain an improved analysis of the OOI. Such feedback information can either occur via optical displays on the scanner 11 or via a monitor connected to the analyzing unit 42 or by means of a projection of light displays onto the surface of the body to be examined.

It shall be understood that for each identified OOI also a corresponding VIS image is stored conveying to the operator an optical impression of the detected decubitus wound or similar. Further processing, display and/or storage of an identified wound is done with known means of electronic data processing.

Moreover, the invention can be used for recognizing bodily infections by local temperature measurement on predetermined body locations. Within the scope of recognition and treatment of patients with a potential virus disease (for example influenza), a rapid and precise measurement of the body temperature of the patient from a distance becomes more and more important. For this purpose, by means of the scanner 11 the body temperature of a potentially infected human being can be measured on the inner eye corners. The scanner 11 has the advantage here that it can capture the head of the patient by means of the three-dimensional recording and subsequently can identify specifically the position of the eyes in the thermal images by correlation with the model stored in the first database 44. By this it is guaranteed that the temperature is actually measured automatically in the eye corners and not, for example, in any other location of the head. If the scanner 11 is, for example, mounted on a stand or tripod, it can be used within an airport sluice for temperature measurement in the eye corners of a large number of airline passengers.

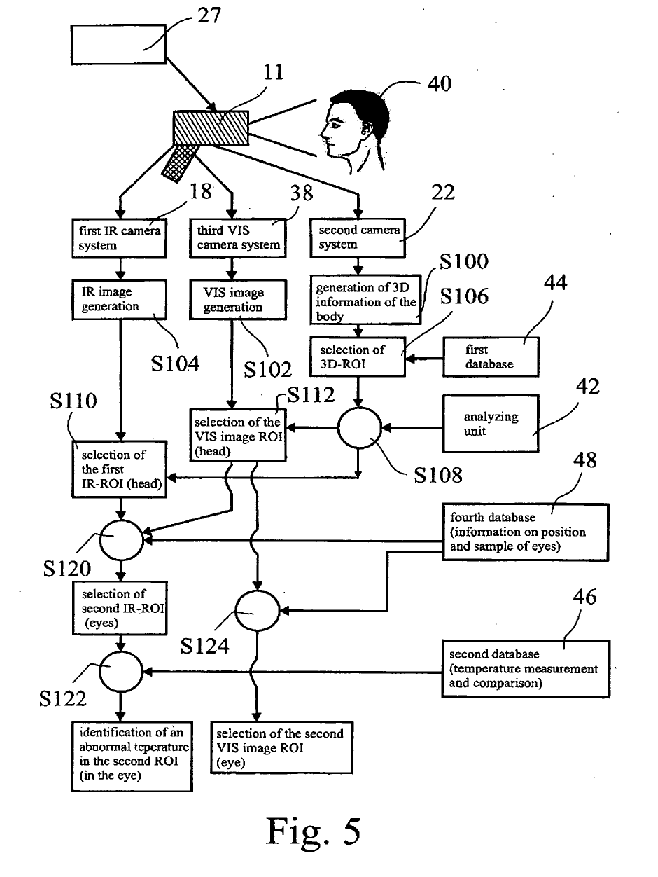

Below by means of FIG. 5 the use of the scanner 11 is described in detail for examination of a patient on a possible disease by a H1N1 or H5N1 infection, also called influenza. FIG. 5 shows in the same way not only the major components of the inventive system but also individual process steps for the use of this system and/or performance of the method. From the upper section of FIG. 5 it becomes clear that the scanner 11 is now directed towards the patient's head 40′ (not the lower leg as in FIG. 4). Up to and including step S112 the scan in FIG. 5 corresponds to that of FIG. 4 so that reference is made to FIG. 4 in order to avoid repetitions.

Examination of the patient for a possible affection by influenza and the corresponding recording of the head 40′ according to FIG. 5 is subdivided into a two step process regarding selection of the ROI of the thermal image taking and also the VIS image of the head. In detail, subsequent to step S110 the Infrared ROI of the head in step S120 is aligned with a fourth database in which information on position and sample of the eyes is stored. This information is linked in that process with the Infrared ROI of the head so that exclusively such pixels of the Infrared ROI of the head are selected corresponding to the area around the eyes. In this way a second Infrared ROI (for the eyes) is created. Subsequently, the second Infrared ROI (for the eyes) is compared with the second database 46 in step S122 where defined temperature data for the eyes are stored. Thus, in accordance with step S116 of FIG. 4 by this comparison an abnormal temperature can be identified in the second Infrared ROI (in the eye and/or in the inner eye corners, as explained above) permitting a conclusion to a possible virus disease.

As in step S122 the ROI of the VIS image of the head is linked in step S124 to the data of the fourth database 48 in order to select a second ROI of the VIS image from it. As a result, thus only such picture elements of the VIS image are taken into account which correspond precisely to the eyes and/or the eye. It shall be understood that in particular such VIS images are stored in respect of which for the corresponding second Infrared ROI a characteristic temperature deviation and thus a disease pattern of the patient (influenza) is identified. Further processing, display and storage of the generated recordings occurs in the same way as in FIG. 4 with known means of electronic data processing.

The invention can likewise be used for measurement of skin or tissue reactions in the case of or after the impact of compressive stress. For this purpose, the patient's skin in a suitable area of the body is for example locally subjected to high mechanical pressure for some time resulting in a discoloration due to a change in perfusion. In parallel, the thermal signature will change in such location. If the pressure is released from this skin location, the tissue requires some time in order to regenerate and reassume its original color and temperature. The time required for it is a guide on the patient's health state. By means of the scanner 11 the time period for this change can be determined by continuous scanning with a real time evaluation with high precision being able to calculate this time period.

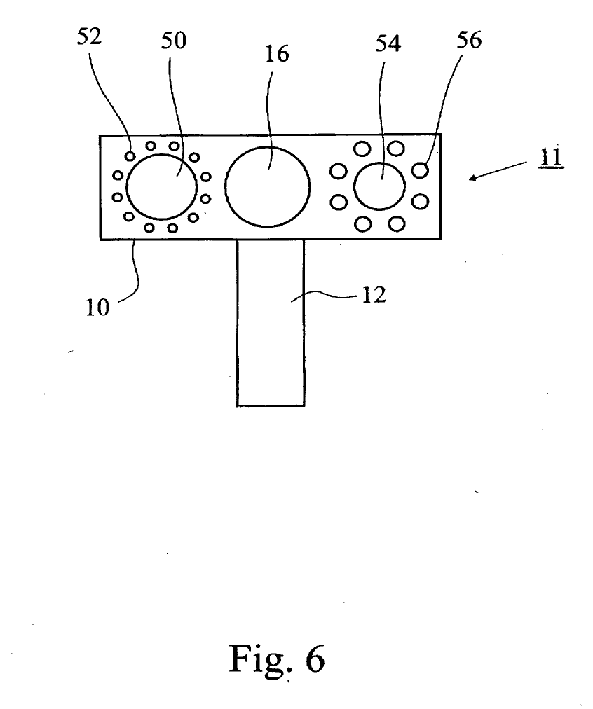

In FIGS. 6-8 another embodiment of the inventive system is shown. Same parts compared to the embodiment of FIG. 1 have the same reference number and are not explained again in order to avoid repetitions.

FIG. 6 is a front view of the front face 14 of the scanner 11. Immediately to the left of the centrally located thermal image camera 16 a so-called time-of-flight (TOF) camera 50 is located. Externally around the TOF camera 50 a plurality of LEDs 52 is located emitting modulated light with a wavelength adjusted to the TOF camera. To the right of the thermal image camera 16 a VIS camera 54 is located which on the perimeter of its lens is surrounded by a plurality of white LEDs 56. The TOF camera 50 shall be understood here as a second camera system 22′ and the VIS camera 54 as a third camera system. Such a third VIS camera system may—contrary to the representation in FIG. 6—also have a plurality of VIS cameras.

The operating principle of the laser 11 according to the embodiment of FIG. 6 corresponds identically to the embodiment of FIG. 1. Contrary thereto, for the scanner 11 in FIG. 6 the second camera system (in the form of the TOF camera 50) and the third VIS camera system (in the form of a single VIS camera 54) are formed as systems separate from each other. Albeit, operation of these camera systems corresponds identically to that of FIG. 1. During recording of a VIS image by means of the VIS camera 56 the white LEDs 56 are switched on so that the body surface is illuminated well for the visual image.

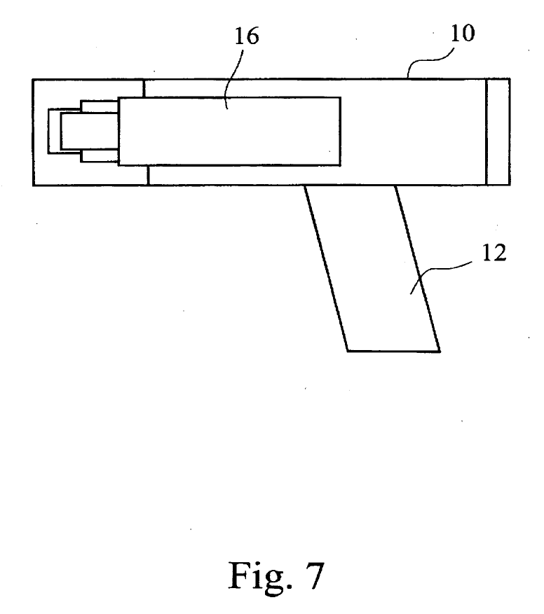

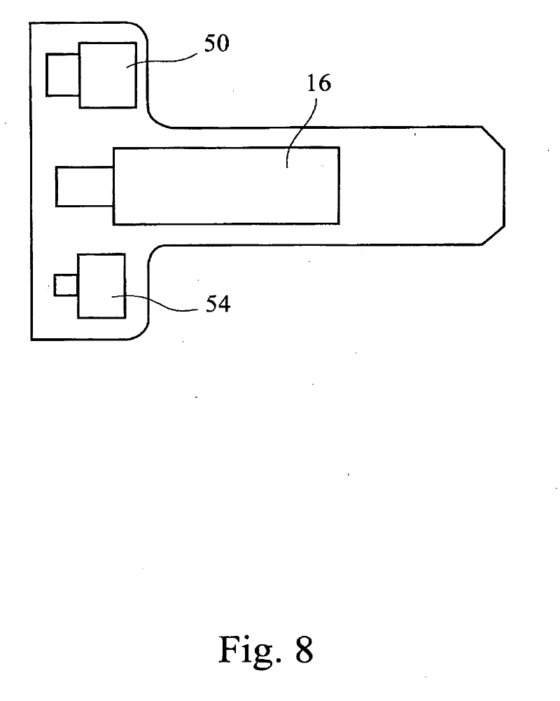

FIGS. 7 and 8 show in the same way as FIGS. 2 and 3 scanner 11 in a lateral sectional view and/or in a longitudinal sectional view. With respect to FIG. 8 it shall be understood that the arrangement of the TOF camera 50 and the VIS camera 54 is shown in simplified symbols here. The TOF camera 50 and the VIS camera 54 are arranged here such that their beam paths extend mainly parallel to each other. Contrary to this also an arrangement of these cameras as shown in FIG. 3 is possible so that the respective beam paths extend towards each other and intersect in point S.

By means of scanner 11 according to the embodiment of FIG. 6-8 a decubitus wound and/or a possible virus disease of a patient can be examined in the same way as explained above by reference to FIGS. 4 and 5. Insofar reference is made to the explanation of FIGS. 4 and 5 in order to avoid repetitions.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A system for detection of a temperature on a surface of a body, the system comprising
a first IR camera system via which the temperature of a certain area of the surface is detectable in the form of a thermal image;
a second camera system via which three-dimensional information is detectable regarding a position of a specific area of the body surface related to the first IR camera system;
at least a first database in which at least for one body a corresponding model is stored by which at least one contour of the body is defined; and
an analyzing unit connectable with the first database receiving the thermal image detected by the first IR camera system and the 3D information detected by the second camera system,
wherein the analyzing unit correlates the 3D information detected by the second camera system with a selectable model stored in the first database thereby generating a data record, which contains exclusively 3D information allocated to the model, the 3D information of the data record subsequently being transformed into a 2D space, and
wherein the transformed 2D information of the generated data record is linkable to the thermal image so that exclusively such pixels of the thermal image are selected corresponding to the 2D information of the data record.

2. The system according to claim 1, further comprising:
a second database in which defined temperature data are stored, and
a recognition module associated with the second database with the temperature data of the selected pixels of the thermal image being comparable to the defined temperature data stored in the second database so that deviations or correlations between the respective temperature data can be determined.

3. The system according to claim 1, further comprising:
a third VIS camera system via which a VIS image of the specific surface area is created,
with the transformed 2D information of the generated data record being linkable to the VIS image so that exclusively such pixels of the VIS image are selected corresponding to the 2D information of the data record.

4. The system according to claim 3, wherein the third VIS camera system is integrated into the second camera system so that via a camera unit of the second camera system a VIS image of the specific surface area is created.

5. The system according to claim 2, further comprising a third database in which the selected pixels of the thermal image, deviations between the respective temperature data of the selected pixels of the thermal image and the defined temperature data stored in the second database and/or the selected pixels of the VIS image can be stored.

6. The system according to claim 1, wherein the first and the second camera system are arranged in a common housing which is configured to be carried by an operator.

7. The system according to claim 1, further comprising a distance measuring device via which a distance between the specific area of the body surface and the first, second, or third camera system is measured.

8. The system according to claim 7, wherein the distance measured between the specific area of the body surface and the first, second, or third camera system is indicated by an acoustic and/or optical signal.

9. The system according to claim 7, wherein the distance measuring device is integrated into the second camera system.

10. The system according to claim 1, wherein the first IR camera system comprises at least one thermal image camera or a plurality of thermal image cameras.

11. The system according to claim 1, wherein the second camera system comprises at least a TOF (time-of-flight) camera and for at least a stereo vision camera.

12. The system according to claim 11, comprising a device for illuminating the specific surface area with a light, a wavelength of which is adjusted to the camera of the second camera system

13. The system according to claim 1, further comprising a device for illuminating a specific surface area with colored or white light which that is detected by a third VIS camera system.

14. The system according to claim 1, further comprising a battery unit providing power supply for individual system components including camera equipment.

15. The system according to claim 1, further comprising an indicating unit which can show the selected pixels of the thermal image and/or the selected pixels of VIS image.

16. The system according to claim 2, wherein the recognition module provides an image processing algorithm via which a specific temperature pattern in the form of an object-of-interest is identified with a signal being representable by an output unit in the event that the object of interest falls below a predetermined distance to a border area of the contour of the transformed 2D data of the data record.

17. The system according to claim 16, wherein the signal in view of a new taking shows a recommended shifting direction for the specific surface area so that the identified object of interest exceeds a predetermined distance to a border area of the contour of the transformed 2D data of the data record.

18. The system according to claim 16, wherein the output unit is integrated into the indicating unit.

19. The system according to claim 1, wherein the creation of temperature recordings of human or animal bodies is possible for medicinal purposes.

20. A method for detection of a temperature on a surface of a body, the method comprising:
detecting the temperature of a specific surface area in the form of a thermal image by a first IR camera system;
detecting three-dimensional information by a second camera system regarding a position of the specific surface area related to the first IR camera system;
selecting a predetermined model, which defines at least a contour of the body and is stored in a first database, and subsequent correlating such model by an analyzing unit with the 3D information of the specific surface area detected by the second camera system, with a data record being generated in which exclusively 3D information is contained which is unequivocally allocable to the model;
transforming the 3D information of the data record into a 2D space; and
linking the transformed 2D information of the data record with the thermal image so that only such pixels of the thermal image are selected which correspond to the 2D information of the data record.

21. The method according to claim 20, further comprising the steps of :
providing defined temperature data which are stored in a second database, and
comparing the temperature data of the selected pixels of the thermal image with the defined temperature data stored in the second database, so that deviations or correlations between the respective temperature data are determined.

22. The method according to claim 20, further comprising the steps of:
creating a VIS image of the specific surface area, and
linking the transformed 2D information of the data record generated by the analyzing unit with the VIS image so that exclusively such pixels of the VIS image are selected which correspond to the 3D information of the data record.

23. The method according to claim 20, wherein a third database is provided in which the selected pixels of the thermal image, deviations between the respective temperature data of the selected pixels of the thermal image and the defined temperature data stored in the second database and/or the selected pixels of the VIS image are stored.

24. The method according to claim 20, wherein a distance between the second camera system and the specific surface area is measured by a distance measuring device with the distance detected being indicated by an acoustic and/or optical signal.

25. The method according to claim 20, wherein the specific surface area is illuminated with coloured light while the temperature of such area is detected by the first IR camera system.

26. The method according to claim 20, wherein the specific surface area is illuminated during detection of the 3D information by the second camera system with a light the wavelength of which is adjusted to the cameras of the second camera system.

27. The method according to claim 20, wherein a specific temperature pattern in the form of an object-of-interest (OOI) is identified in the specific area of the body surface by an image processing algorithm with a distance of the OOI to a border area of the contour of the transformed 2D data of the data record being compared with a predetermined value, and an alarm signal being generated, if such distance falls below the predetermined value.

28. The method according to claim 27, wherein the alarm signal in view of a new recording of the specific surface area shows a recommended shifting direction for the first IR camera system and/or the second camera system so that the distance of the OOI to the border area of the contour of the transformed 2D data of the data record exceeds a predetermined distance.

29. The method according to claim 20, wherein the method serves for creating temperature takings of human or animal bodies for medicinal purposes.

30. The method according to claim 29, wherein, via which an analysis of wound areas of the skin surface of human or animal bodies is carried out.

31. The method according to claim 20, wherein an information regarding spatial orientation of the body is generated with such information being taken into account for correlating the model with the 3D information of the specific area of the body surface detected by the second camera system.
